(19) United States
(12) Patent Application Publication
(10) Pub. No.: US 2013/0331286 A1
(43) Pub. Date: Dec. 12, 2013

(54) UNIVERSAL RANDOM ACCESS DETECTION OF NUCLEIC ACIDS

(71) Applicant: Christopher C. Sappenfield, Carlsbad, CA (US)

(72) Inventor: Christopher C. Sappenfield, Carlsbad, CA (US)

(73) Assignee: IBIS BIOSCIENCES, INC., Carlsbad, CA (US)

(21) Appl. No.: 13/907,281

(22) Filed: May 31, 2013

Related U.S. Application Data
(60) Provisional application No. 61/653,585, filed on May 31, 2012.

(51) Int. Cl. C12Q 1/68

(52) U.S. Cl. CPC C12Q 1/6844
USPC 506/9; 506/39

(57) ABSTRACT

Provided herein is technology relating to detecting nucleic acids in a sample and particularly, but not exclusively, to systems and methods related to random access primer pair libraries that are used to configure or customize assays for nucleic acid detection.

### CROSS-REFERENCE TO RELATED APPLICATIONS

The present application claims priority to U.S. Provisional Application Ser. No. 61/653,585 filed May 31, 2012, the entirety of which is incorporated by reference herein.

### FIELD OF TECHNOLOGY

Provided herein is technology relating to detecting nucleic acids in a sample and particularly, but not exclusively, to systems and methods related to random access primer pair libraries that are used to configure or customize assays for nucleic acid detection.

### BACKGROUND

The polymerase chain reaction (PCR) is a primer-directed in vitro reaction for the enzymatic amplification of a specific DNA fragment. Saiki, “Enzymatic Amplification of β-Actin Genomic Sequences and Restriction Site Analysis for Diagnosis of Sickle Cell Anemia”, Science 230: 1350-54 (1985). PCR is generally considered the most sensitive and rapid method for detecting nucleic acids in a particular sample. PCR is well-known in the art and has been described, e.g., in U.S. Pat. No. 4,683,195 to Mullis et al.; U.S. Pat. No. 4,683,202 to Mullis; U.S. Pat. No. 5,298,392 to Atlas et al.; and U.S. Pat. No. 5,437,990 to Burg et al. In PCR, an oligonucleotide primer pair for each target is provided wherein each primer pair includes a first nucleotide sequence complementary to a sequence flanking the 5 prime end of the target nucleic acid sequence and a second nucleotide sequence complementary to a nucleotide sequence flanking the 3 prime end of the target nucleic acid sequence. The nucleotide sequences of each oligonucleotide primer pair are specific to a particular target sequence or sequences to be detected and are designed not to cross-react with other non-target sequences.

The distinctive nature of the PCR process in producing a substantive quantity of DNA fragments of interest from an initial tiny amount of DNA sample has gained broad application in the fields of biomedical research and clinical diagnosis. For example, PCR has been widely used in the diagnosis of inherited disorders, the individualization of evidence samples in the forensics area, and the detection of bacterial and viral pathogens and potential bioterror agents. See, e.g., Erlich et al, “Recent Advances in the Polymerase Chain Reaction”, Science 252: 1643-51 (1991); Newton & Graham, PCR (Oxford, 1994); Sontakke, “Use of broad range16S rDNA PCR in clinical microbiology”, J Microbiol Methods 76: 217-25 (2009); Yang, “PCR-based diagnostics for infectious diseases: uses, limitations, and future applications in acute-care settings” Lancet Infect Dis 4: 337-48 (2004); Sninsky, “The polymerase chain reaction (PCR): a valuable method for retroviral detection”, Lymphology 23: 92-7 (1990); Fykse, “Detection of bioterror agents in air samples using real-time PCR”, J Appl Microbiol 105: 351-8 (2008).

For example, PCR has played a critical role in genotyping a vast number of genetic polymorphisms and individual variations which underlie the onset of many diseases, see, e.g., Shi, “Enabling Large-Scale Pharmacogenetic Studies by High-throughput Mutation Detection and Genotyping Technologies”, Clin Chem 47: 164-172 (2001), and forms part of standard laboratory tests to detect clinically relevant pathogens, see e.g., Riffelmann, “Nucleic Acid Amplification Tests for Diagnosis of Bordetella Infections”, J Clin Microbiol 43: 4925-4929 (2005).

Widespread applications notwithstanding, the use of PCR is quite often limited by the costs and time associated with designing and assembling PCR assays. At the initial stages, selecting a target typically involves bioinformatic analysis of known sequences to identify sequences specific for the required detection. Then, providing a template nucleic acid comprising the target for amplification involves choosing a molecular biological method appropriate for the source of the nucleic acid and applying it to the sample. For example, an environmental sample and a cultured bacterial isolate may involve using different protocols and reagents for preparing quality template. The PCR assay itself involves designing, selecting, and synthesizing oligonucleotide primers that will robustly and reproducibly amplify the target without, e.g., amplifying non-target sequences or forming primer dimers and/or hairpins. Assembling a reaction requires providing target nucleic acid, nucleotides, primers, polymerase, buffers, and other components at the appropriate concentrations in a reaction vessel. Experiments can easily involve hundreds and thousands of individual reactions, each one requiring a precise measurement and delivery of these components into the appropriate reaction vessel. Performing the thermocycling of the PCR requires selecting and/or programming a series of temperature cycles that are tuned to the melting, annealing, and extension of the particular template(s) and primers in the reaction as well as the buffers, salts, and other components of the reaction. Finally, the resulting amplicon may require purification before detection and evaluation by a chosen detection method. For example, some applications may use a probe to determine if an amplicon is present, while some applications may use sequencing to provide more information about mutations, strain variation, etc., at single-nucleotide resolution. As each of these steps often requires validation, testing, and appropriate experimental controls, developing, performing, and evaluating the results of a PCR assay can be demanding on the attention and time of researchers already having limited resources. Moreover, user proficiency and knowledge of molecular biology, enzyme biochemistry, data analysis, etc., at an expert level is often required for the assay.

Some conventional technologies have been developed in an attempt to address some of these issues. For example, multi-channel pipettors, multititer plates, and other parallel fluid handling systems have made some aspects of PCR assay assembly more reliable and less time-consuming. Also, premixed solutions, e.g., of polymerase, buffer, and nucleotides have reduced some fluid handling and mixing steps. In addition, bioinformatic tools have made target selection and primer design more systematic for the user.

### SUMMARY

Accordingly, provided herein is technology related to detecting nucleic acids in a sample. The systems include integrated “Assay Factories” and/or random access primer pair libraries that are used to configure or customize assays for particular detection pathways in essentially real-time. In some embodiments, the systems are configured for universal detection and identification (e.g., to detect essentially any nucleic acid in a given sample); in some embodiments systems are configured for something less than universal detection and identification (e.g., human genetic-based diagnostics/prognostics, pathogen only diagnostics, etc.). The technology provides serial and/or parallel reaction schemes depending on user input that improve time-to-answer, flexibility, cost effectiveness (e.g., pinpoint charge-per-well capability) and automation. The technology finds use, e.g., in molecular diagnostics and prognostics.

For example, some embodiments of the technology provide a system for identifying a nucleic acid, the system comprising a random access primer component configured to provide a primer; a nucleic acid amplification component configured to amplify a nucleic acid using the primer to generate an amplicon; and an amplicon detection component configured to detect a property of the amplicon. In some embodiments, the system comprises an expert system that aids a user in, e.g., developing assays and/or interpreting results; thus, in some embodiments, the systems according to the technology further comprise an expert system. In some embodiments, the system further comprises a conveyance component configured to convey the primer from the random access primer component to the amplification component and/or configured to convey the amplicon from the nucleic acid amplification component to the amplicon detection component. In addition, some embodiments provide a system that further comprises a controller operably connected to one or more of the random access primer component, the nucleic acid amplification component, the amplicon detection component, and/or the conveyance component and configured to effect one or more of conveying the primers from the random access primer component to the nucleic acid amplification component, conveying the amplicon from the nucleic acid amplification component to the amplicon detection component, amplifying the nucleic acid with the nucleic acid amplification component, and/or detecting a property of the amplicon with the amplicon detection component.

In some embodiments, the primers are stored in the random access primer component and in some embodiments the primers are synthesized in the random access primer component. Thus, some embodiments provided that the random access primer component comprises a random access primer library and/or an oligonucleotide synthesis component. In addition, in some embodiments of the systems, the nucleic acid amplification component comprises a thermocycler component. In some embodiments, the amplicon detection component comprises a mass spectrometer component, a fluorescence detection component, and/or a nucleic acid sequencing component.

The technology is not limited in the technology that is used to detect and/or characterize the amplicon. For example, in some embodiments, one or more properties of the amplicon is/are measured, e.g., its presence and/or absence, mass, partial base composition, complete base composition, partial sequence, complete sequence, hybridization to a probe, electrophoretic mobility, length, hydrodynamic character, and restriction pattern.

The random access primer component is configured to provide a primer pair in some embodiments of the systems. The technology is not limited in the size and/or capacity of the random access primer component. As such, the random access primer component comprises 10 to 1000 primers, approximately 25, 50, 75, 100, 200, 300, 400, 500, 600, 700, 800, or 900 primers. In some embodiments, the random access primer component comprises more than 1000 primers.

In some embodiments, the nucleic acid amplification component is configured to pre-amplify a nucleic acid, e.g., to provide sufficient template for amplification. The technology encompasses embodiments of systems comprising a sample preparation component configured to receive a sample and prepare the nucleic acid from the sample. Furthermore, embodiments of the systems comprise a database, wherein the database comprises a sample preparation protocol, a pre-amplification protocol, a primer datum, an amplification program, an amplicon detection protocol, and/or a reference amplicon property. In some embodiments, data for primers is stored in a database, e.g., in some embodiments, primer data relating to a primer nucleotide sequence, a primer name, a primer location in the random access primer component, a primer melting temperature, and/or a primer target is/are stored in an database.

In some embodiments, reference data are stored in a database. For example, in some embodiments, data relating to one or more properties of a reference amplicon are stored in a database, e.g., a reference amplicon property such as the presence or absence of the amplicon, mass, partial base composition, complete base composition, partial sequence, complete sequence, hybridization to a probe, electrophoretic mobility, length, hydrodynamic character, and/or a restriction pattern.

Some embodiments of the systems comprise a modular random access vessel, e.g., in some embodiments, a modular random access vessel assembled into a reaction pathway. In some embodiments, the systems comprise a vessel scaffold and/or a reagent storage component. The technology also provides embodiments of the systems in which the expert system comprises a knowledge base, wherein the knowledge base comprises rules for selecting an assay to detect a nucleic acid.

The technology finds use, e.g., for the detection and characterization of a nucleic acid, e.g., for the detection and characterization of an organism, cell, tissue, chromosome, gene, SNP, and/or individual.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a schematic drawing showing an exemplary embodiment of a system architecture related to the technology provided herein.

FIG. 2 is a schematic drawing showing a random access reaction vessel. FIG. 2A shows a partially transparent top view of a modular random access reaction vessel. FIG. 2B shows a partially transparent first side view of a modular random access reaction vessel. FIG. 2C shows a partially transparent back view of a modular random access reaction vessel. FIG. 2D shows a partially transparent front view of a modular random access reaction vessel. FIG. 2E shows a partially transparent second side view of a modular random access reaction vessel. FIG. 2F shows a partially transparent perspective view of a modular random access reaction vessel. FIG. 2G shows the assembly of multiple modular random access reaction vessels to provide an assembled reaction pathway.

FIG. 3 is a schematic drawing showing the assembly of modular random access reaction vessels. FIG. 3A shows a top view of a modular random access reaction vessel. FIG. 3B shows a partially transparent side view of the modular random access reaction vessel shown in FIG. 3A. FIG. 3C shows a top view of three modular random access reaction vessels assembled to provide a reaction pathway. FIG. 3D shows a top view of eight modular random access reaction vessels assembled to provide a reaction pathway. FIG. 3E shows a top view of sixteen modular random access reaction vessels assembled to provide a 2×8 reaction pathway. FIG. 3F shows a partially transparent side view of the 2×8 reaction pathway shown in FIG. 3E. FIG. 3G shows a top view of ninety-six modular random access reaction vessels assembled to provide a 12×8 reaction pathway (e.g., in a 96-well plate configuration). FIG. 3H is a side view of a scaffold or reaction container support structure. FIG. 3I is a sectional side view of a scaffold or reaction container support structure without any positioned reaction containers. FIG. 3J is a sectional side view of a scaffold or reaction container support structure showing positioned reaction containers and reaction containers being positioned.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to detecting nucleic acids in a sample and particularly, but not exclusively, to systems and methods related to random access primer pair libraries that are used to configure or customize assays for nucleic acid detection.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products (“amplicons”) are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

The term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N⁶-methyladenosine, aziridinylcytosine, pseudoisocytosine, 5-(carboxyhydroxyl-methyl)-uracil, 5-fluorouracil, 5-bromouracil, 5-carboxymethylaminomethyl-2-thiouracil, 5-carboxymethyl-aminomethyluracil, dihydrouracil, inosine, N⁶-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N⁶-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

It is well known that DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. It is also known that all of these 5 types of nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

The term “communicate” refers to the direct or indirect transfer or transmission, and/or the capability of directly or indirectly transferring or transmitting, something at least from one thing to another thing. Objects “fluidly communicate” with one another when fluidic material is, or is capable of being, transferred from one object to another. Objects are in “thermal communication” with one another when thermal energy is or can be transferred from one object to another. Objects are in “magnetic communication” with one another when one object exerts or can exert a magnetic field of sufficient strength on another object to effect a change (e.g., a change in position or other movement) in the other object. Objects are in “sensory communication” when a characteristic or property of one object is or can be sensed, perceived, or otherwise detected by another object. It is to be noted that there may be overlap among the various exemplary types of communication referred to above.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

“Nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N-6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, La.), all herein incorporated by reference in their entireties.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68:90-99; the phosphodiester method of Brown et al. (1979) Meth Enzymol. 68:109-151; the diethylphosphoramidite method of Beaucage et al. (1981) Tetrahedron Lett. 22:1859-1862; the triester method of Matteucci et al. (1981) J Am Chem Soc 103:3185-3191; automated synthesis methods; or the solid support method of U.S. Pat. No. 4,458,066, or other methods known to those skilled in the art. All of these documents are incorporated by reference.

A “polymerase” is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase 1, Klenow fragment, Thermophilus aquaticus DNA polymerase, Tth DNA polymerase, Vent DNA polymerase (New England Biolabs), Deep Vent DNA polymerase (New England Biolabs), Bst DNA Polymerase Large Fragment, Stoeffel Fragment, 9° N DNA Polymerase, Pfu DNA Polymerase, Tfl DNA Polymerase, RepliPHI Phi29 Polymerase, Tli DNA polymerase, eukaryotic DNA polymerase beta, telomerase, Therminator polymerase (New England Biolabs), KOD HiFi DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase, novel polymerases discovered by bioprospecting, and polymerases cited in US 2007/0048748, U.S. Pat. Nos. 6,329,178, 6,602,695, and 6,395,524 (incorporated by reference). These polymerases include wild-type, mutant isoforms, and genetically engineered variants.

As used herein a “sample” refers to anything capable of being analyzed by the methods and systems provided herein. In some embodiments, the sample comprises or is suspected to comprise one or more nucleic acids capable of analysis by the methods. In certain embodiments, for example, the samples comprise nucleic acids (e.g., DNA, RNA, cDNAs, etc.) from one or more organisms, tissues, or cells. Samples can include, for example, blood, semen, saliva, urine, feces, rectal swabs, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “solid support” is a solid material having a surface for attachment of molecules, compounds, cells, or other entities. The surface of a solid support can be flat or not flat. A solid support can be porous or non-porous. A solid support can be a chip or array that comprises a surface, and that may comprise glass, silicon, nylon, polymers, plastics, ceramics, or metals. A solid support can also be a membrane, such as a nylon, nitrocellulose, or polymeric membrane, or a plate or dish and can be comprised of glass, ceramics, metals, or plastics, such as, for example, polystyrene, polypropylene, polycarbonate, or polyallomer. A solid support can also be a bead, resin or particle of any shape. Such particles or beads can be comprised of any suitable material, such as glass or ceramics, and/or one or more polymers, such as, for example, nylon, polytetrafluoroethylene, TEFLON, polystyrene, polyacrylamide, sepaharose, agarose, cellulose, cellulose derivatives, or dextran, and/or can comprise metals, particularly paramagnetic metals, such as iron.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

A “system” denotes a set of components, real or abstract, comprising a whole where each component interacts with or is related to at least one other component within the whole. For example, a “system” in the context of analytical instrumentation refers a group of objects and/or devices that form a network for performing a desired objective.

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “target” refers to a nucleic acid sequence or structure to be detected or characterized.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, modular random access vessel, etc.).

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid, oligonucleotide, or polynucleotide may be present in single-stranded or double-stranded form.

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “signal” as used herein refers to any detectable effect, such as would be caused or provided by a label or an assay reaction.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof.

### Embodiments of the Technology

Provided herein is technology relating to detecting nucleic acids in a sample and particularly, but not exclusively, to systems and methods related to random access primer pair libraries that are used to configure or customize assays for nucleic acid detection.

Some embodiments of the technology comprise a system of components, e.g., a random access primer component, a nucleic acid amplification component, an amplicon detection component, a conveyance component, a controller, a sample preparation component, and/or a database. Particular embodiments comprise various combinations of two or more of these components.

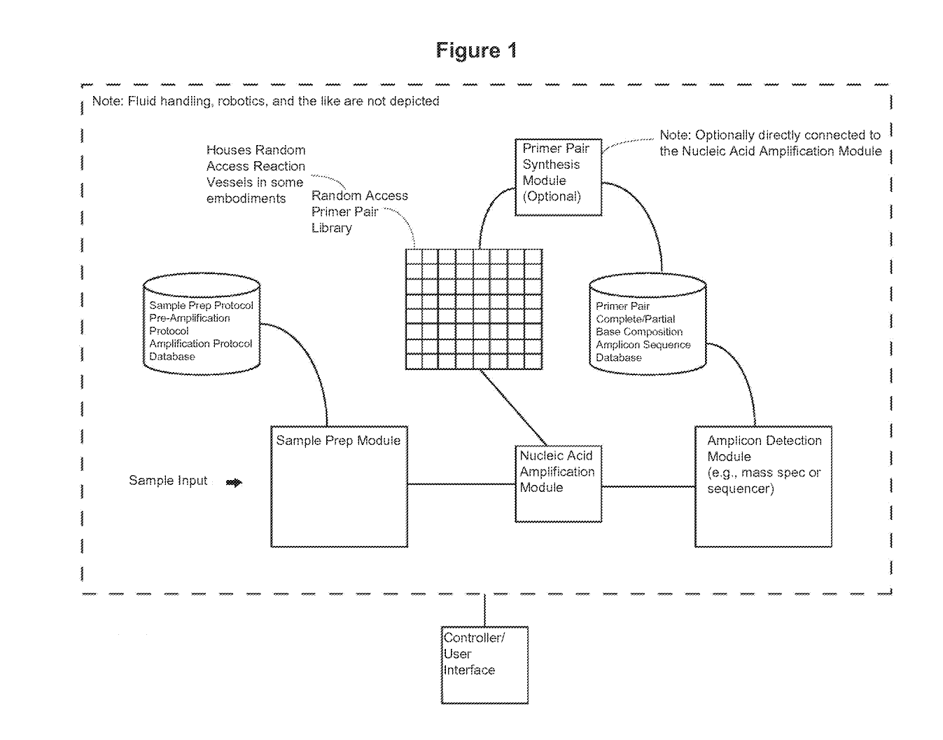

For example, FIG. 1 shows an embodiment of the technology comprising several components. First, a sample preparation component receives a sample and prepares a nucleic acid from the sample. In some embodiments, the sample and/or the nucleic acid is prepared according to a sample preparation protocol stored in a database. Furthermore, some embodiments optionally comprise use of pre-amplification of the nucleic acid according to a pre-amplification protocol stored in a database. Then, the nucleic acid is transported (e.g., by a conveyance component) to a nucleic acid amplification module for amplification using one or more reagents from a reagent storage component and PCR primers from a random access primer pair library or, optionally, by primers synthesized on-demand by a primer pair synthesis component. Information about the PCR primers (e.g., sequence, melting temperature, position in the primer library, etc.) is stored in a primer database and the nucleic acid in the sample is amplified according to an amplification protocol (e.g., a thermal cycling program) stored in a database. After amplification, the amplified nucleic acid is evaluated by an amplicon detection component comprising in some embodiments a nucleic acid sequencer and/or a mass spectrometer. The data collected from the amplicon is assessed using data collected in a reference database such as a base composition database, a sequence database, etc. A controller coordinates and integrates the components of the system and a user interface comprises a functionality for a user to provide information to the system and for the system to provide information to the user.

This is one illustrative embodiment of the technology and is not intended to be limiting. The scope of the technology is developed in more detail according to the description of these and other components that find use in various embodiments of the technology as discussed below.

Random Access Primer Component

In some embodiments the technology comprises a random access primer component. The random access primer component is configured to store (e.g., in a random access vessel as described below) and/or synthesize primers for PCR and make them available for use, e.g., in the nucleic acid amplification component. Accordingly, in embodiments of the technology the random access primer component comprises one or more primer storage vessels (e.g., a random access vessel) in which is stored one or more primers (e.g., a single primer or a primer pair or sets of primer pairs). In addition, embodiments provide that the storage vessels comprise an oligonucleotide storage solution, e.g., comprising one or more of a buffer, a salt, a preservative, and/or other components that are appropriate to provide a composition (e.g., a solution) for the stable storage of oligonucleotides to minimize or eliminate the degradation of the oligonucleotides (e.g., to inhibit enzymatic and/or chemical breakdown). Some embodiments provide that the random access primer component is temperature controlled to maintain the primer solutions at, below, or above a particular temperature. For example, in some embodiments the primer solutions are kept as a liquid (e.g., are not frozen) at a temperature above the freezing point of the primer solutions, e.g., at approximately 4° C. In some embodiments, the primer solutions are kept in a frozen state, e.g., at or below approximately −20° C. or at or below approximately −80° C., and are thawed to provide a portion for distribution, e.g., to the nucleic acid amplification component, and use for the technology. In some embodiments, the primers are stored at a concentration that is ready for use when mixed with other components of a PCR.

In some embodiments, the primers are stored as a PCR pre-mix, e.g., with all components required for a PCR (e.g., primers, salts, polymerase, buffer, nucleotides (e.g., dNTPs, e.g., dATP, dCTP, dGTP, and dTTP) except the sample template. In such embodiments, the pre-mix is a single use composition to which sample template is added for thermal cycling and amplification. Such premixes are stored, in some embodiments, in random access vessels for use according to the technology provided herein.

In some embodiments, the primers are stored at individually addressable locations. For instance, in some embodiments each storage location has a unique address that is stored in a database associated with other data and information (e.g., nucleotide sequence and physical characteristics) for the primer or primers stored at that address and location. The address is used in some embodiments to place a primer at a storage location and/or to access a storage location to provide a primer for an assay. Accordingly, the random access primer component is configured such that any primer in the component can be accessed, e.g., to provide a primer for an assay.

In some embodiments, the random access primer component comprises an oligonucleotide synthesis component. As used herein, the term “oligonucleotide synthesis component” refers to a component of a system that is capable of synthesizing oligonucleotides. For example, the oligonucleotide synthesis component in some embodiments synthesizes an oligonucleotide on demand such that the oligonucleotide has a sequence for use as a PCR primer, e.g., to amplify a target by the nucleic acid amplification component. In some embodiments a synthesized oligonucleotide is synthesized, optionally purified, and then used for amplification (e.g., in the nucleic acid amplification component), while in some embodiments a synthesized oligonucleotide is synthesized, optionally purified, then stored in the random access primer component (e.g., in a primer storage vessel, e.g., a random access vessel as described below), e.g., for later use in an amplification (e.g., in the nucleic acid amplification component). Technology for automated synthesis of oligonucleotides is described, e.g., in U.S. Pat. Appl. Pub. No. 20080261220, which is incorporated herein by reference for all purposes.

The present technology is not limited to any one type of synthesizer. Indeed, a variety of synthesizers are contemplated, including, but not limited to MOSS EXPEDITE 16-channel DNA synthesizers (PE Biosystems, Foster City, Calif.), OligoPilot (Amersham Pharmacia), the 3900 and 3948 48-Channel DNA synthesizers (PE Biosystems, Foster City, Calif.), POLYPLEX (Genemachines), 8909 EXPEDITE, Blue Hedgehog (Metabio), MerMade (BioAutomation, Plano, Tex.), Polygen (Distribio, France), PrimerStation 960 (Intelligent Bio-Instruments, Cambridge, Mass.), and the high-throughput synthesizer described in PCT Publication WO 01/41918. In some embodiments, synthesizers are modified or are wholly fabricated to meet physical or performance specifications particularly preferred for use in the synthesis component of the present invention. In certain embodiments, the synthesizers are configured for generating oligonucleotides in 96 or 384 well plates, or in the modular reaction or storage vessels as described herein. In some embodiments, the oligonucleotide synthesis component further comprises an automated reagent supply system, e.g., as described in U.S. Pat. Appl. Pub. No. 20080261220, incorporated herein by reference in its entirety for all purposes.

Some embodiments provide that the synthesized oligonucleotides have sequences defined on an ad hoc basis by a user for a particular assay or that are generated on an ad hoc basis by computer software to be appropriate for amplifying a particular target. For example, a user may enter the desired nucleotide sequence of the oligonucleotide using a combination of characters (e.g., A, C, G, and T) entered by an input device such as a keyboard. In some embodiments, nucleotide sequences for primers are stored in a database and provided to the oligonucleotide synthesis component. In some embodiments, the oligonucleotide synthesis component comprises stocks of nucleotides that serve as monomers in an oligonucleotide synthesis. For example, the nucleotides in some embodiments comprise the bases adenine, thymine, cytosine, and guanosine. Some embodiments provide that the oligonucleotide synthesis component comprises stocks of nucleotides that comprise non-standard bases such as inosine, xanthine, modified bases (e.g., iso-C, iso-G), and other base variants known in the art.

In some embodiments, the random access primer component and/or the oligonucleotide synthesis component comprises an oligonucleotide purification and/or an oligonucleotide processing component. For example, embodiments provide an oligonucleotide processing component that is capable of processing oligonucleotides post-synthesis. Examples of oligonucleotide processing include, but are not limited to, purification, drying, cleavage and deprotection, desalting, dilution and filling, and quality control. Components configured to perform these functions are described in, e.g., U.S. Pat. Appl. Pub. No. 20080261220, incorporated herein by reference in its entirety for all purposes). Embodiments provide that an oligonucleotide purification component removes particular components of an oligonucleotide storage solution and/or an oligonucleotide synthesis reaction, e.g., that may act as inhibitors of an amplification reaction in the nucleic acid amplification component. For example, some embodiments remove unincorporated nucleotides and/or chemicals associated with oligonucleotide synthesis from the primers as is appropriate for their use in the nucleic acid amplification component. Some embodiments remove a cryoprotectant from an oligonucleotide storage solution that increases the stability of a stored oligonucleotide but that may also reduce the efficiency of an amplification reaction.

Reagent Storage Component

Some embodiments provide a reagent storage component for storing (e.g., stored in a modular random access vessel) general reagents used in the sample preparation component; the nucleic acid amplification component; the oligonucleotide synthesis component; and/or to process, purify, or isolate amplicons. Examples of reagents that are stored in the reagent storage component include distilled deionized water, detergents (e.g., SDS, Triton X-100) alcohols (e.g., 2-propanol, ethanol, methanol, phenol), organic solvents (e.g., chloroform, acetonitrile), buffers (e.g., Tris-HCl, (NH₄)₂SO₄), chelators (e.g., EDTA), salts (e.g., MgCl₂, KCl, MnCl₂), enzymes (e.g., polymerases, lysozymes, proteases, etc.), nucleotides (e.g., dNTPs such as dATP, dCTP, dGTP, and dTTP), nucleotide mixtures, labels (e.g., fluorescent or mass labels), premixed PCR reagents (e.g., a composition comprising more than one PCR component), and other components of general use for molecular biology such as beta-mercaptoethanol, bovine serum albumin, salmon sperm DNA, acids, bases, tRNA, etc. In some embodiments the random access primer component comprises the reagent storage component.

Modular Random Access Vessels

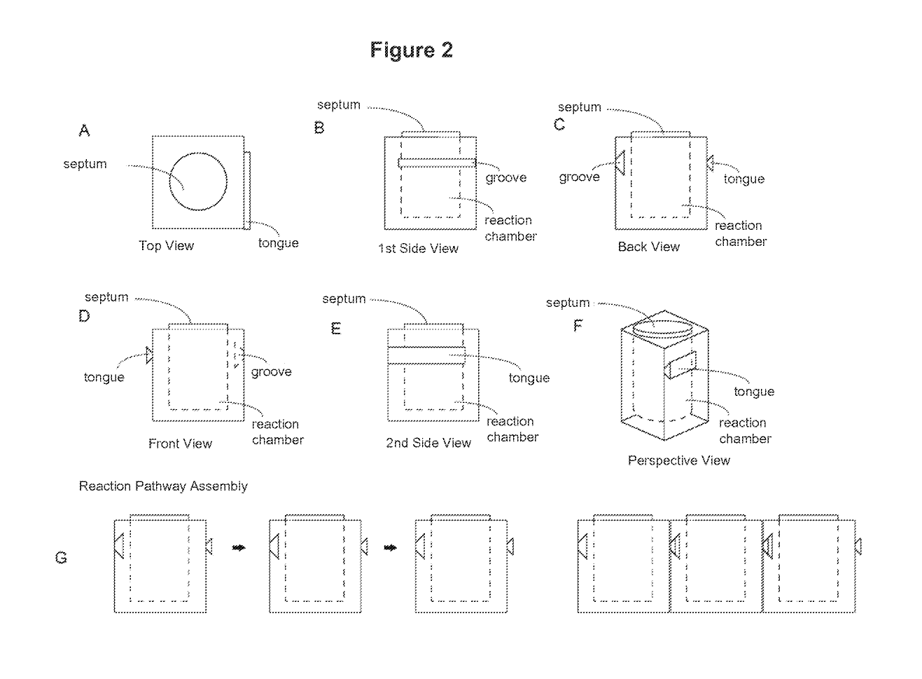

Some embodiments comprise modular random access vessels for the storage, transport, and reaction of assay components. FIG. 2 shows schematic drawings of an embodiment of a modular random access vessel. In some embodiments (e.g., as shown in FIG. 2), the vessels are shaped approximately as a rectangular prism or approximately as a cube and have a circular hole on the top face for access to the vessel contents. In some embodiments, the vessels comprise a septum on the top surface that covers the hole but is able to be breached (e.g., punctured, passed through) by an instrument such as a pipette tip, a needle, a sampler, a cannula, a tube, etc. for access to the vessel contents. In some embodiments, the vessels are for a single use; however, in some embodiments, the vessels are re-filled, re-sealed with a septum, and re-used in the system.

In some embodiments, the vessels comprise a tongue and groove on opposite sides that provide for mechanically linking vessels together, e.g., to provide an array of vessels (e.g., a linear array such as an 8-vessel strip, a rectangular array such as a 2×8 or an 8×12 array, or any configuration appropriate for the assays to be performed).

The vessels are made of any suitable material such as plastic, metal, rubber (e.g., silicone), etc. that are appropriate for the assays to be performed (e.g., PCR). In some embodiments, the vessels are made from a plastic such as polycarbonate, cyclo-olefin copolymer, cyclo-olefin polymer, polystyrene, polymethylmethacrylate, polypropylene, polyethylene, or some other polyolefinpolypropylene. In some embodiments, the vessels are transparent, e.g., so that they may find use as a cuvette for spectrometric or fluorometric analysis. In some embodiments, the vessels are opaque, e.g., so that they protect light sensitive chemicals and reagents stored within the vessel from exposure to light. In some embodiments, the vessels (whether transparent, translucent, opaque, semi-opaque, etc.) are colored, e.g., some shade or variation of black, white, red, orange, yellow, green, blue, or violet. In some embodiments, the colors are used as part of a color code, e.g., to signify the contents of the vessel, to signify the material from which the vessel is made, to signify the status of an assay in the vessel, to signify a size of the vessel, etc.

Embodiments of the vessels have dimensions from approximately 1 mm to 10 mm, from 10 mm to 100 mm, from 100 mm to 1 cm, and/or from 1 to 10 cm or more.

In some embodiments, the vessels conform to ANSI standards for microtiter plates with respect to the dimensions and positions of the wells for microtiter plates having 48, 96, 384, and 1536 wells. These involve ANSI/SBS Standards 1-2004 through 4-2004, as well as Standard SBS-5 currently under development, the entire contents of which are incorporated herein by reference. In some embodiments, configurations having any number of vessels less than 96 vessels conform to these standards as they apply to individual wells/vessels and the relative arrangement and positioning of one vessel with respect to neighboring vessels.

The modular random access vessel is used by the system as a universal vessel, e.g., to store a reagent or reagent mixture; to assemble a reaction (e.g., for collecting the reaction components and mixing them); to act as a reaction vessel (e.g., to hold the reaction while it is thermally cycled); to act as a sample holder for analysis and/or detection (e.g., to hold the reaction product, to act as a cuvette, to provide aliquots for sampling, etc.); to store reactions and/reaction products for long-term storage; to archive reaction components, input samples (e.g., nucleic acids prepared from a sample to be analyzed), reaction products, etc.; to transport reaction components, reaction products, reaction intermediates, samples, nucleic acids, etc. amongst components of the system as well as, in some aspects, outside the system (e.g., to send through the mail and/or using other modes of shipping said entities).

A brief summary of an exemplary assay illustrates the uses of the vessels according to the technology. In one aspect of the technology, random access storage vessels store buffers, salts, enzymes (e.g., polymerases), nucleotides, nucleotide mixtures, premixed PCR reagents, etc. As such, according to the technology, the various components for a PCR assay are selected from a collection of reagents, each stored in a vessel, and transported (e.g., by the conveyance component) in the storage vessels and/or moved to a transport vessel for transport. The contents of the vessels are used to assemble and mix the required assay reaction(s) in one or more reaction vessels, which may be an empty vessel or one of the vessels holding a reaction component (e.g., the vessel holding a buffer). In some embodiments the components are stored together as a pre-mixed solution comprising the PCR assay components except a sample template.

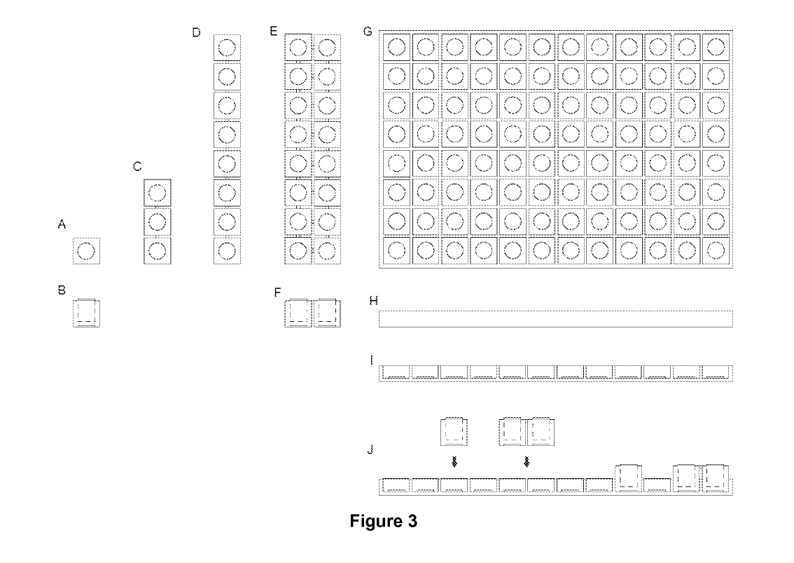

For example, an illustrative embodiment of using the vessels in a simple assay designed to detect one particular antibiotic-resistant bacterium comprises a PCR to detect a signature gene for that bacterium (e.g., targeting a 16S rRNA gene), a PCR to detect one or more antibiotic resistance genes (e.g., targeting a beta-lactamase, a drug efflux pump, an acetylase, etc.), and one or more negative and/or positive controls. For this particular exemplary assay, the system is designed to select and transport vessels holding, e.g., 1) a primer pair for detecting the particular 16S rRNA, 2) a primer pair for detecting a drug resistance gene, 3) a nucleotide solution of dATP, dCTP, dGTP, and dTTP, 4) an enzyme solution comprising, e.g., Taq polymerase, 5) a reaction buffer comprising appropriate salts, and 6) a target template (e.g., as provided by the sample preparation component). The various test and control PCRs are assembled by the system into empty vessels or, alternatively, into one of the vessels holding a reaction component (e.g., a vessel holding reaction buffer). These reaction vessels are then assembled into a reaction pathway by linking the vessels to one another (see FIGS. 2 and 3) and transporting (e.g., by the conveyance component) the assembled reaction pathway to the nucleic acid amplification component for thermal cycling. The vessels are then transported to the amplicon detection component for analysis, e.g., by fluorimetry, sequencing, and/or mass spectrometry. The reactions are analyzed while in the vessels (e.g., by detecting fluorescence emission and using the vessel as a cuvette) and/or an aliquot is removed for analysis (e.g., by mass spectrometry). The resulting data is acquired and processed (e.g., by using software bioinformatic and/or database tools) and results provided to the user.

In some embodiments, the vessels are assembled upon a scaffold (also known as a reaction container support structure or a vessel support structure) as shown in FIG. 3. The scaffold is in the form of a tray having features appropriate to mate with a vessel and hold it in place. While FIG. 3 shows an embodiment of a scaffold configured in a 9×12 array (e.g., in the form of a 96-well plate), the technology is not limited in the configuration of the scaffold. The scaffold technology is intended to comprise a structure having any arrangement of vessel mating structures.

The scaffolds are made of any suitable material such as plastic, metal, rubber (e.g., silicone), cardboard, wood, laminates of these materials, etc. that are appropriate for the assays to be performed. In some embodiments, the scaffolds are made from a plastic such as polycarbonate, cyclo-olefin copolymer, cyclo-olefin polymer, polystyrene, polymethylmethacrylate, polypropylene, polyethylene, or some other polyolefinpolypropylene. In some embodiments, the scaffolds are transparent and in some embodiments, the scaffolds are opaque. In some embodiments, the scaffolds (whether transparent, translucent, opaque, semi-opaque, etc.) are colored, e.g., some shade or variation of black, white, red, orange, yellow, green, blue, or violet. In some embodiments, the colors are used as part of a color code, e.g., to signify the contents of a vessel mated with the scaffold, to signify an assay to be performed using the scaffold, etc.

Nucleic Acid Amplification Component

In one aspect, the technology comprises a nucleic acid amplification component. The nucleic acid amplification component effects the amplification of a nucleic acid, e.g., by polymerase chain reaction (PCR), reverse transcription polymerase chain reaction (RT-PCR), transcription-mediated amplification (TMA), ligase chain reaction (LCR), strand displacement amplification (SDA), and/or nucleic acid sequence based amplification (NASBA; see, e.g., U.S. Pat. No. 5,130,238, incorporated herein by reference in its entirety). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA). For further discussion of known amplification methods see, e.g., Persing, David H., “In Vitro Nucleic Acid Amplification Techniques” in Diagnostic Medical Microbiology: Principles and Applications (Persing et al., eds.), pp. 51-87 (American Society for Microbiology, Washington, D.C. (1993)).

For example, essentially any thermal cycling station or device is optionally adapted for use with embodiments of the technology. Examples of suitable thermocycling devices that are optionally utilized are available from many different commercial suppliers, including Mastercycler devices (Eppendorf North America, Westbury, N.Y., U.S.A.), the COBAS AMPLICOR Analyzer (Roche Molecular Systems, Inc., Pleasanton, Calif., U.S.A.), Mycycler and iCycler Thermal Cyclers (Bio-Rad Laboratories, Inc., Hercules, Calif., U.S.A.), and the SmartCycler System (Cepheid, Sunnyvale, Calif., U.S.A.), among many others. In other exemplary embodiments, sample preparation components, nucleic acid amplification components, and related fluid handling or material transfer components (e.g., a conveyance component) are integrated with the systems described herein, e.g., to fully automate a given nucleic acid amplification and analysis process. Instruments that can be adapted for this purpose include, for example, the m2000 automated instrument system (Abbott Laboratories, Abbott Park, Ill., U.S.A.), the GeneXpert System (Cepheid, Sunnyvale, Calif. U.S.A.), and the COBAS AmpliPrep System (Roche Molecular Systems, Inc., Pleasanton, Calif., U.S.A.), and the like.

The nucleic acid amplification component comprises one or more reaction chambers where an amplification reaction occurs. The reaction chamber may hold the reaction components (e.g., buffer, nucleotides, target, primers, enzyme, etc.) directly or the reaction chamber may hold a modular random access vessel in which the reaction components reside.

Amplicon Detection Component

In one aspect, the technology comprises an amplicon detection component. In some embodiments, the amplicon detection component comprises a detector. Detectors are typically structured to detect detectable signals produced, e.g., in or proximal to another component of the given assay system (e.g., in a container, e.g., a modular random access vessel and/or on a solid support). Suitable signal detectors that are optionally utilized, or adapted for use, herein detect, e.g., fluorescence, phosphorescence, radioactivity, absorbance, refractive index, luminescence, or mass. Detectors optionally monitor one or a plurality of signals from upstream and/or downstream of the performance of, e.g., a given assay step. For example, detectors optionally monitor a plurality of optical signals, which correspond in position to “real-time” results. Example detectors or sensors include photomultiplier tubes, CCD arrays, optical sensors, temperature sensors, pressure sensors, pH sensors, conductivity sensors, or scanning detectors. Detectors are also described in, e.g., Skoog et al., Principles of Instrumental Analysis, 5th ed., Harcourt Brace College Publishers (1998), Currell, Analytical Instrumentation: Performance Characteristics and Quality, John Wiley & Sons, Inc. (2000), Sharma et al., Introduction to Fluorescence Spectroscopy, John Wiley & Sons, Inc. (1999), Valeur, Molecular Fluorescence: Principles and Applications, John Wiley & Sons, Inc. (2002), and Gore, Spectrophotometry and Spectrofluorimetry: A Practical Approach, 2nd ed., Oxford University Press (2000), which are each incorporated by reference.

Amplicons can be detected by any conventional means. For example, in some embodiments, nucleic acids are detected by hybridization with a detectably labeled probe and measurement of the resulting hybrids. Illustrative non-limiting examples of detection methods are described below. One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention.

In some embodiments, the base compositions of amplicons are determined from detected molecular masses. In these embodiments, base compositions are typically correlated with the identity of an organismal source, genotype, or other attribute of the corresponding template nucleic acids in a given sample. Suitable software and related aspects, e.g., for determining base compositions from detected molecular masses and for performing other aspects of base composition analysis are commercially available from Ibis Biosciences, Inc. (Carlsbad, Calif., U.S.A.). Nucleic acid base composition analysis is also described in, e.g., U.S. Pat. Nos. 7,255,992; 7,226,739; 7,217,510; and 7,108,974, which are each incorporated by reference in their entireties.

For example, in some embodiments intact molecular ions are generated from amplicons using one of a variety of ionization techniques to convert the sample to the gas phase. These ionization methods include, but are not limited to, electrospray ionization (ESI), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, the present technology provides nucleic acid sequencing methodologies and/or technologies. Nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc. Aspects of nucleic acid sequencing platforms and associated computer systems are described, e.g., in U.S. Pat. Appl. Pub. No. 20110270533, incorporated herein by reference in its entirety.

In some embodiments, sequencing methodologies and technologies comprise traditional or first generation sequencing technologies (Maxam & Gilbert, 1977, Proc Natl Acad Sci USA 74: 560-564; Sanger et al., 1977, Proc Natl Acad Sci USA 74: 5463-5467; herein incorporated by reference in their entireties) that utilize electrophoretic detection on a gel or through capillary electrophoresis ((Smith et al., 1986, Nature 321: 674-679; herein incorporated by reference in its entirety). In some embodiments, DNA sequencing methodologies provided by the present technology comprise Second Generation (a.k.a. Next Generation or Next-Gen), Third Generation (a.k.a. Next-Next-Gen), or Fourth Generation (a.k.a. N3′ Gen) sequencing technologies, including but not limited to pyrosequencing, sequencing-by-ligation, single molecule sequencing, sequence-by-synthesis (SBS), massively parallel clonal, massively parallel single molecule SBS, massively parallel single molecule real-time, massively parallel single molecule real-time nanopore technology, etc. Morozova and Marra provide a review of some such technologies in Genomics 92:255 (2008), herein incorporated by reference.

For example, in some embodiments, the present technology provides DNA sequencing by pyrosequencing (Ronaghi et al. 1998, Science 281:363, 365; Ronaghi et al. 1996, Analytical Biochemistry 242: 84; Nyren 2007, Methods Mol Biology 373: 1-14; herein incorporated by reference in their entireties). Pyrosequencing is a method of DNA sequencing based on the “sequencing by synthesis” principle, which relies on detection of pyrophosphate release. “Sequencing by synthesis” involves immobilizing a single strand of the DNA, and synthesizing its complementary strand enzymatically. The pyrosequencing method is based on detecting the activity of DNA polymerase with a chemiluminescent enzyme. Pyrosequencing allows sequencing of a single strand of DNA by synthesizing the complementary strand along it, one base pair at a time, and detecting which base added at each step. The template DNA is immobilized, and solutions of A, C, G, and T nucleotides are added and removed after the reaction, sequentially. Chemiluminescence is produced when the nucleotide solution complements the next unpaired base of the template. The sequence of solutions which produce chemiluminescent signals provides sequence of the template.

In some embodiments, the present technology provides DNA sequencing by 454 sequencing as developed by ROCHE LIFE SCIENCES. In 454 sequencing, SBS pyrosequencing is performed in polony beads in picoliter-scale wells, provides very long read lengths (400-500 bases), and can yield approximately 400-600 Mbases/run or 1 billion bases/day. 454 sequencing finds utility in de novo sequencing, resequencing, expression tags, transcriptome sequencing, ChIP, methylation analysis, etc. 454 sequencing involves annealing of ssDNA to an excess of DNA capture beads, emulsification of beads and PCR reagents in water-in-oil microreactors, clonal amplification, breaking of microreactors, and enrichment for DNA positive beads. 454 sequencing is performed on a GENOME FLX SEQUENCER.

In some embodiments, the present technology provides DNA sequencing by SOLiD sequencing as developed by APPLIED BIOSYSTEMS. SOLiD sequencing utilizes polony-based sequencing methodologies (Mitra & Church 1999 Nucleic Acids Res, 27:e34; herein incorporated by reference in its entirety). Polony sequencing provides a nonelectrophoretic sequencing method without in vivo cloning artifacts at a low cost per base. In some embodiments, an in vitro paired-tag library is constructed from genomic DNA. Library molecules are clonally amplified on microbeads by emulsion PCR, the clonal amplification yields polymerase colonies, or polonies, that can be sequenced. Short reads are generated in parallel from the microbeads via a cyclic DNA sequencing strategy that utilizes T4 DNA ligase to selectively tag each microbead with fluorescent labels that correlate with the unique nucleotide sequence present on any given bead. SOLiD sequencing provides sequencing by ligation using T4 DNA ligase, fluorescent-labeled degenerate nonamers, “Two Base Encoding” which provides increased accuracy (>99.94%), read length up to 35 bases, and high throughput of 20 Gb/run. SOLiD sequencing finds utility in de novo sequencing, targeted and whole genome resequencing, gene expression, transcriptome and methylation analysis. SOLiD sequencing is performed on a SOLiD 3 platform.

In some embodiments, the present technology provides DNA sequencing by ILLUMINA sequencing technology. ILLUMINA sequencing technology utilizes massively parallel SBS using reverse terminator chemistry. SBS is performed at 4 bases/cycle versus 1 base/cycle for pyrosequencing. ILLUMINA sequencing relies on the attachment of randomly fragmented genomic DNA to a planar, optically transparent surface. Attached DNA fragments are extended and bridge amplified to create an ultra-high density sequencing flow cell with 80-100 million clusters, each containing 1,000 copies of the same template. These templates are sequenced using a four-color DNA SBS technology that employs reversible terminators with removable fluorescent dyes. In some embodiments, high-sensitivity fluorescence detection is achieved using laser excitation and total internal reflection optics. ILLUMINA sequencing provides read lengths of up to 75 bases, throughput of approximately 10-15 Gb/run, and a paired end strategy allows sequencing from both ends. ILLUMINA sequencing finds utility in de novo sequencing, resequencing, transcriptome analysis, epigenomic/methylation status. ILLUMINA sequencing is performed on a GENOME ANALYZER platform.

In some embodiments, the present technology provides DNA sequencing by TRUE SINGLE MOLECULE SEQUENCING (TSMS) by HELICOS BIOSCIENCES. TSMS provides massive parallel single molecule SBS using 1 base per cycle of pyrosequencing. TSMS does not require any up-front library synthesis steps or PCR amplification, therefore eliminating PCR errors. TSMS relies on attachment of billions of single molecules of sample DNA on an application-specific proprietary surface. The captured strands serve as templates for the sequencing-by-synthesis process in which polymerase and one fluorescently labeled nucleotide (C, G, A or T) are added, polymerase catalyzes the sequence-specific incorporation of fluorescent nucleotides into nascent complementary strands on all the templates, free nucleotides are removed by washing, incorporated nucleotides are imaged and positions recorded, the fluorescent group is removed in a highly efficient cleavage process leaving behind the incorporated nucleotide, and the process continues through each of the other three bases. Multiple four-base cycles result in complementary strands greater than 25 bases in length synthesized on billions of templates, providing a greater than 25-base read from each individual template. TSMS provides very high density arrays (1 million/mm²), low cost/base, two laser system (Cy3 and Cy5-labeled dNTP), and read lengths of ˜20-55 bases. TSMS finds utility in human genome resequencing, de novo sequencing. TSMS is performed on the HELISCOPE platform.

In some embodiments, the present technology provides DNA sequencing by VISIGEN BIOTECHNOLOGIES. VISIGEN BIOTECHNOLOGIES sequencing provides massive parallel single molecule sequencing in real-time through engineered DNA polymerases and nucleoside triphosphates which function as direct molecular sensors of DNA base identity. Genetically engineered polymerase is fixed on the surface during synthesis. Fluorescence resonance energy transfer (FRET) is detected between the immobilized polymerase and labeled dNTP as they are incorporated. VISIGEN sequencing provides no up-front amplification or cloning steps, read lengths of 1,000 bases, massive parallel arrays (1 Mb/sec/instrument), and no sequential reagent addition during synthesis. VISIGEN sequencing finds utility in de novo sequencing, resequencing, personalized medicine, clinical diagnostics, forensics, basic research, etc.

In some embodiments, the present technology provides single molecule real time (SMRT) sequencing by PACIFIC BIOSCIENCES. SMRT provides massive parallel single molecule sequencing in real-time. Thousands of zero-mode waveguides (ZMWs) in zeptoliter wells are contained on an array. A single DNA polymerase molecule is attached to the bottom of each waveguide. DNA is synthesized using .gamma.-phosphate group labeled with base-specific fluorophores. Upon incorporation of a phospholinked nucleotide, the DNA polymerase cleaves the dye molecule from the nucleotide when it cleaves the phosphate chain. Fluorophores are detected upon incorporation of the corresponding base by the immobilized polymerase. SMRT provides low reaction volumes, very low fluorescence background, fast cycle times, with long read lengths (approx. 1,000 bases), and no sequential reagent addition during synthesis. SMRT find utility in de novo sequencing, resequencing, etc.

In some embodiments, the Xpandomer technology of STRATOS is used (see e.g., U.S. Pat. Pub. No. 20090035777, herein incorporated by reference in its entirety). In this approach, methods for sequencing a target nucleic acid comprise providing a daughter strand produced by a template-directed synthesis, the daughter strand comprising a plurality of subunits coupled in a sequence corresponding to a contiguous nucleotide sequence of all or a portion of the target nucleic acid, wherein the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand, the Xpandomer comprising the tethers and reporter elements for parsing genetic information in a sequence corresponding to the contiguous nucleotide sequence of all or a portion of the target nucleic acid. Reporter elements of the Xpandomer are then detected.

In some embodiments, “four-color sequencing by synthesis using cleavable fluorescents nucleotide reversible terminators” as described in Turro, et al. PNAS 103: 19635-40 (2006) is used, e.g., as commercialized by Intelligent Bio-Systems. The technology described in U.S. Pat. Appl. Pub. Nos. 2010/0323350, 2010/0063743, 2010/0159531, 20100035253, 20100152050, incorporated herein by reference for all purposes.

In some embodiments, nanopore sequencing is used in which integrated circuits enable massively parallel single-molecule DNA sequencing, e.g., as described in Rothberg, 2011 “An integrated semiconductor device enabling non-optical genome sequencing”, Nature 475:348; Timp, 2010 “Nanopore Sequencing-Electrical Measurements of the Code of Life”, IEEE Transactions on Nanotechnology 9:281; Stoddart & Hagan Bayley et al., 2009 “Single-nucleotide discrimination in immobilized DNA oligonucleotides with a biological nanopore”, PNAS 106:7703. Such technology is commercialized by Genia.

In some embodiments, sequencing is performed using a two-base degenerate code (see, e.g., U.S. Pat. Appl. Ser. No. 61/641,715, incorporated herein by reference in its entirety for all purposes). For example, rather than determining the sequence of the four bases A, C, G, and T in a nucleic acid, some embodiments of the technology determine the order of purine and pyrimidine bases in a nucleic acid. Using a sequencing scheme according to this exemplary approach, the conventionally derived sequence ACGT would instead be acquired by determining that the sequence consists of a purine in the first position, a pyrimidine in the second position, a purine in the third position, and a pyrimidine in the fourth position, which may be represented as RYRY. An alternative two-base sequencing scheme based on identifying the sequence of keto bases and amino bases produces the sequence of MMKK for this same four-base sequence of bases ACGT. In some embodiments, the information of the two two-base sequences can be merged to produce a conventional four-base sequence. According to the current example, the first position is an amino purine base, the second position is an amino pyrimidine base, the fourth position is a keto purine base, and the fourth position is a keto pyrimidine base, which leads unambiguously to the sequence ACGT.

Some embodiments provide that the sequencing is performed using a sequencing-by-synthesis approach in which differences in signal amplitude rather than differences in signal wavelength (e.g., color) are used to identify each base incorporated during a sequencing reaction (see, e.g., U.S. Pat. Appl. Ser. No. 61/641,718, incorporated herein by reference in its entirety for all purposes). In this scheme, each individual base is labeled with the same moiety (e.g., a dye, a fluorescent label, etc.) at a different known percentage (e.g., a “label fraction” or “extent of labeling”). As an exemplary embodiment, 25% of the ATP molecules are labeled, 50% of the TTP molecules are labeled, 75% of the GTP molecules are labeled, and 100% of the CTP molecules are labeled. Then, according to some embodiments, an ensemble (e.g., a polony or a clonal colony) based sequencing approach is performed and the sequence is determined by detecting a signal intensity after each base incorporation and associating the intensities with the bases.

In some embodiments, the sequencing technology relies on differences in labeling ratios rather than on only differences in color to identify bases incorporated during a sequencing reaction (see, e.g., U.S. Pat. Appl. Ser. No. 61/641,720, incorporated herein by reference in its entirety for all purposes). In this scheme, each individual nucleotide base is labeled at a specific known ratio of at least two different moieties (e.g., a dye, a fluorescent label, etc.). As an exemplary embodiment, ATP is labeled with two moieties X and Y in a ratio of 1:0 (all ATP molecules are labeled with moiety X), TTP is labeled with the two moieties X and Yin a ratio of 2:1 (two-thirds of the population of TTP molecules is labeled with moiety X and one-third of the population of TTP molecules is labeled with moiety Y), GTP is labeled with the two moieties X and Y in a ratio of 1:2 (one-third of the population of GTP molecules is labeled with moiety X and two-thirds of the population of GTP molecules is labeled with moiety Y), and CTP is labeled with the two moieties X and Y in a ratio of 0:1 (all CTP molecules are labeled with moiety Y). Then, according to some embodiments, a polony (e.g., a clonal colony) based sequencing approach is performed with the sequence determined by detecting the ratio of signals produced by the two dyes after each base incorporation.

Conveyance Component

Embodiments of the technology comprise a conveyance component. The conveyance component is configured to move modular random access vessels, assemblies of modular random access vessels (e.g., a reaction pathway), reagents, reaction components, reaction products, sensors, detectors, system components, etc. For example, the conveyance component in some embodiments transports a primer and/or reagents from the random access primer component to the nucleic acid amplification component. In some embodiments, the conveyance component transports an amplicon from the nucleic acid amplification component to the amplicon detection component. Exemplary technologies that find use in the conveyance component include fluidics (e.g., microfluidics), mechanical devices (e.g., belts, chains, gears, wires, robotics), hydraulics, pneumatics, etc.

In some embodiments, the conveyance component is configured to convey a modular vessel as described herein. For example, in some embodiments the conveyance component comprises a grasping component to grasp a modular vessel and a transport component to transport the modular vessel to a location where it is needed for an assay. As another example, some embodiments provide that the conveyance component comprises a track component for transporting the modular vessel. In some embodiments the conveyance component is configured to transport a scaffold or reaction vessel support structure (e.g., as shown in FIG. 3). In some embodiments, the conveyance component is configured to transport and/or operate automatic pipettors, needles, and other devices for fluid acquisition, transport, and/or delivery.

Controller

In one aspect, the technology comprises a controller. A controller is typically operably connected to one or more of the other components and is generally configured to effect various functionalities of the various components, e.g., the assembly and mixing of a PCR assay, the movement of containers, the movement of carrier mechanisms (e.g., a conveyance component), the transfer of materials, the detection of one or more property of an amplicon, and the like. Controllers are typically operably connected to one or more system components, such as motors (e.g., via motor drives), thermal modulating components, detectors, motion sensors, fluidic handling components, robotic translocation devices, or the like, to control operation of the components. More specifically, controllers are generally included either as separate or integral system components. Controllers and/or other system components is/are generally coupled to an appropriately programmed processor, computer, digital device, or other logic device or information appliance (e.g., including an analog to digital or digital to analog converter as needed), which functions to instruct the operation of these instruments in accordance with preprogrammed or user input instructions (e.g., primer selection, fluid volumes to be conveyed, etc.), receive data and information from these instruments, and interpret, manipulate and report this information to the user.

A controller or computer optionally includes a monitor which is often a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation. The computer then receives the data from, e.g., sensors/detectors included within the system, and interprets the data, either provides it in a user understood format, or uses that data to initiate further controller instructions, in accordance with the programming, e.g., such as in monitoring detectable signal intensity, rates or modes of sample processing unit container rotation, or the like.

More specifically, the software utilized to control the operation of the sample processing stations and systems of the technology typically includes logic instructions. The logic instructions of the software are typically embodied on a computer readable medium, such as a CD-ROM, a floppy disk, a tape, a flash memory device or component, a system memory device or component, a hard drive, a data signal embodied in a carrier wave, and/or the like. Other computer readable media are known to persons of skill in the art. In some embodiments, the logic instructions are embodied in read-only memory (ROM) in a computer chip present in one or more system components, without the use of personal computers.

The computer can be, e.g., a PC (e.g., an Intel x86 or Pentium chip-compatible computer comprising DOS, OS2, WINDOWS, LINUX, MACINTOSH, Power PC, or a UNIX (e.g., SUN) software) or other common commercially available computer which is known to one of skill. Standard desktop applications such as word processing, spreadsheet, and database software can be adapted to the present technology. Software for performing, e.g., sample preparation, reaction mixing, amplicon detection, data analysis, etc., is optionally constructed by one of skill using a standard programming language such as Visual basic, C, C++, Fortran, Basic, Java, or the like.

The components optionally include detectors or detection components configured to detect one or more detectable signals or parameters from a given process or composition, e.g., from materials in the random access primer component, amplification component, etc. In some embodiments, systems are configured to detect detectable signals or parameters that are upstream and/or downstream of a given process. Suitable signal detectors that are optionally utilized in these systems detect, e.g., pH, temperature, pressure, density, salinity, conductivity, fluid level, radioactivity, luminescence, fluorescence, phosphorescence, molecular mass, emission, transmission, absorbance, and/or the like. In some embodiments, the detector monitors a plurality of signals. Example detectors or sensors include PMTs, CCDs, intensified CCDs, photodiodes, avalanche photodiodes, optical sensors, scanning detectors, or the like. Each of these as well as other types of sensors is optionally readily incorporated into the components and systems described herein. The detector optionally moves relative to the components or, alternatively, the components move relative to the detector. Optionally, the components and systems include multiple detectors, e.g., that are placed either in or adjacent to, e.g., one or more components such that the detector is in sensory communication with the component (e.g., the detector is capable of detecting a property of the component or a portion thereof, the contents of a portion of the component, or the like, for which that detector is intended).

The detector optionally includes or is operably linked to a computer, e.g., which has system software for converting detector signal information into assay result information or the like. For example, detectors optionally exist as separate units, or are integrated with controllers into a single instrument. Integration of these functions into a single unit facilitates connection of these instruments with the computer, by permitting the use of a few or even a single communication port for transmitting information between system components. Detection components that are optionally included in the systems of the technology are described further in, e.g., Skoog et al., Principles of Instrumental Analysis, 6th Ed., Brooks Cole (2006) and Curren, Analytical Instrumentation: Performance Characteristics and Quality, John Wiley & Sons, Inc. (2000), which are both incorporated by reference.

Sample Preparation Component

In some embodiments of the technology disclosed, the systems comprise a sample preparation component. In some embodiments, a sample is exposed to appropriate reagents to release (e.g., lyse) nucleic acid from cells, tissues, or other sample types. In some embodiments, capture components or molecules (e.g., columns, resins, beads, capture probes, etc.) are used to isolate the nucleic acid from the non-nucleic acid components of the sample. Any of a wide variety of nucleic acid isolation or capture technologies may be used in the sample preparation component of the systems, devices, and methods.

In some embodiments, cell capture technologies are used to isolate cells or other materials (e.g., a virus) containing a target nucleic acid away from other cells and sample material. In other embodiments, Si-pillar arrays are used to capture cells (see, e.g., Hwang et al., Anal. Chem., 80:7786 (2008), herein incorporated by reference in its entirety). For example, in some embodiments, ADEMTECH VIRO ADEMBEADS are used for magnetic separation of viral particles.

In some embodiments, cell lysis comprises using a chemical (e.g., chaotropic salts, GITC, guanidinium-HCl, urea, phenol, NaOH/KOH, detergents, etc.), temperature (boiling, freeze/thaw, microwave), physical force (e.g., pressure, bead beating, French press, sonication, grinding, mortar/pestle/SiO₂), an enzyme (e.g., lysozyme, glycanase, protease, Proteinase K), or osmosis (e.g., osmotic shock, low salt buffers), or combinations thereof. Lysis can be organisms-specific or non-organisms-specific.

Nucleic acid isolation from lysed cellular material or other materials can be conducted by Solid Phase Reversible Immobilization using magnetic microparticles (see e.g., U.S. Pat. No. 5,234,809, herein incorporated by reference in its entirety). In some embodiments, capture oligonucleotides complementary to a target nucleic acid of interest are employed.

Some embodiments of the sample preparation component comprise magnets to facilitate certain processing steps that involve magnetically-based separation of materials. In some embodiments, electromagnets are used and in some embodiments permanent magnets are used.

Database Component

In an aspect, the technology comprises one or more databases to store information, for example:
1. Protocols and methods database, e.g.:
   a. sample preparation (e.g., isolating cells and/or nucleic acids) from a variety of sample types (cells, tissues, environmental samples, cultures, etc.);
   b. pre-amplification protocols (e.g., whole genome amplification);
   c. PCR assay assembly (e.g., identities, concentrations, amounts, masses, etc. of reaction components);
   d. oligonucleotide synthesis, purification, and isolation;
   e. thermocycling programs and parameters (e.g., temperatures, times, temperature ramp parameters, number of cycles);
   f. amplicon clean-up (e.g., desalting) and/or purification (e.g., removing unincorporated nucleotides);
   g. detection assays (e.g., mass spectrometry, sequencing, fluorescence detection);
   h. bioinformatics (e.g., sequence searches, sequence validation, base composition calculations, etc.)
2. Sequence databases, e.g.:
   a. primer sequences (and addresses, characteristics of primers, etc.) of primers stored in the random access primer component;
   b. primer sequences for synthesis;
   c. human genome sequence;
   d. GenBank, EMBL, NCBI, 16S rRNA, bacterial genome sequences, and other public sequence databases;
   e. private sequence databases (e.g., genome sequences of, e.g., pathogens generated in-house or on a contract basis);
   f. amplicon sequences (e.g., amplicons expected for particular detection assays);
   g. SNP database;
   h. complete/partial base compositions;
3. Mass spectrometry databases (e.g., ions, fragments, peaks, peak spectra, etc.)
4. Assay databases
   a. assay targets, genes, organisms, strains, SNPs, amplicons;
   b. primers for particular targets;
   c. reaction conditions for particular targets and primers;
5. Expert system databases
   a. knowledge base
   b. words, phrases, and natural language components for dialog interaction with users;
   c. IF . . . THEN and question and answer databases;
   d. interface components;
6. Operational control databases
   a. users;
   b. limits, alarms, operational variables of the system;
   c. logging and reporting;
   d. reagent and modular vessel inventory;
   e. controller code, subroutines, component-associated software packages.

Bioinformatic Analysis Component

Bioinformatic processing utilizes one or more sequence and information databases (e.g., public or private sequence databases) and software applications for processing sequence and database information. In some preferred embodiments, databases and software for in silico analysis are housed in a single location on one or more computers. Housing the databases and processing software locally provides increased and consistent speed and access to information. In other embodiments, one or more databases and software components located on external computers are accessed over a communication network (e.g., accessed over the World Wide Web).

In some embodiments, bioinformatic processing compares test amplicons to an amplicon database, e.g., by comparing sequences, mass spec data, base compositions, etc. In some embodiments, bioinformatic processing identifies an organism or strain (e.g., at one or more general and/or specific taxonomic levels, e.g., ranging from kingdom to substrain or isolate), gene, tissue, plasmid, chromosome, SNP, allele, mutation, individual, gender, virus, nucleic acid, e.g., by comparing a measured characteristic of an amplicon (sequence, mass spec data, base composition, etc) to a database of characteristics. In some embodiments, the bioinformatic processing provides a qualitative answer (e.g., presence/absence) and in some embodiments the bioinformatic processing provides a quantitative answer (e.g., how many copies, organisms, etc.).

In some embodiments, the target is known and either present in a database of the present technology or provided by the user. In some embodiments, an appropriate target sequence (and thus primers) is/are not known prior to designing and performing the detection assay. Accordingly, some embodiments provide for the analysis of a nucleic acid sequence to identify an appropriate target or primer pair for that particular nucleic acid sequence or organism, gene, tissue, genome, chromosome, etc. having that particular nucleic acid sequence. For example, in some embodiments analyzing a nucleic acid sequence to identify target regions and/or primers comprises preliminary sequence screening (e.g., identifying repeats, low complexity regions, artifact (e.g., vector) sequences), searching a database, processing database information, etc. Targets are presented to the user for selection.

In some embodiments, the user is given the option to select another target sequence or to proceed with the present target sequence. In some embodiments, when problems are identified, the systems of the present invention automatically select and test additional candidate target sequences based on the original requested candidate target sequence (e.g., select neighboring sequences and/or remove problem portions of the sequence). If more reliable sequences are identified, these suggested alternate target sequences are reported to the user. In addition, in some embodiments, the selection of primers to amplify the target is performed in an automated fashion (e.g. by a software application such as Primer3, Primer Prim'er, LAMP, PrimerDesigner, eper, Unifrag, SBEPrimer, or others known in the art). In some embodiments, primer design comprises picking putative candidate primers using software such as Primer3, finding all partial and complete matches of the candidate primer sequence on the target by, e.g., BLAST, FASTA, etc., predicting the amplification products of all possible primer combinations (e.g., using a thermodynamic model of PCR), and searching a set of compatible primers that are specific to the target loci.

In some embodiments, the methods and systems described herein are associated with a programmable machine designed to perform a sequence of arithmetic or logical operations as described herein (e.g., to provide an expert system). For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data (e.g., in a knowledge base). In some embodiments, a microprocessor is part of a system for nucleic acid detection, e.g., a system comprising one or more of a CPU, a graphics card, a user interface (e.g., comprising an output device such as display and an input device such as a keyboard), a storage medium, and memory components. Memory components (e.g., volatile and/or nonvolatile memory) find use in storing instructions and/or data (e.g., a work piece such as a target sequence, amplicon sequence, primer sequence, etc.; a knowledge base; a protocol; etc.). Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to transmitting data over a network (e.g., a local area network (LAN), a wide area network (WAN), an ad-hoc network, etc.). In some embodiments, programmable machines are present on such a network as peers and in some embodiments the programmable machines have a client/server relationship.

In some embodiments, data (e.g., in a database) are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to analyze a nucleic acid (e.g., identify targets, identify primers, perform bioinformatic analysis, query a database, etc.), e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, and JavaScript.

Computers are connected in some embodiments to a network or, in some embodiments, can be stand-alone machines. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. In some embodiments, MATLAB provides a programming environment suitable for performing embodiments of the methods provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device. All such components, computers, and systems described herein as associated with the technology may be logical or virtual.

Expert System Component

In some embodiments, the technology comprises an expert system having a simple user interface, flexible and specialized learning knowledge bases, data structures and process for storing user tested protocol methods, hierarchy of parameter selection rules, measurement unit conversion tools, robust experimental design and analysis tools, display of the experiment design analysis in a way which is easily understood, and an optional feedback method for the refinement of the protocol method. In some embodiments, an expert system comprises aspects of artificial intelligence (AI), e.g., a computer program that can simulate the judgment and behavior of a human or an organization that has expert knowledge and experience in a particular field. Thus, in some embodiments, the technology comprises an expert system that is a computer system that emulates the decision-making ability of a human expert. See, e.g., Jackson, Peter (1998), Introduction to Expert Systems (3 ed.), Addison Wesley.

In some embodiments, the expert system comprises an inference engine, a knowledge base, and a dialog interface to communicate with users. The knowledge base (also known as a rule base) contains accumulated experience and a set of rules for applying the knowledge base to each particular situation that is described to the program. Another expert system is known as neuronal network (NN) which is capable of actively accumulating information and knowledge. Other expert systems are well known to those of skill in the art and need not be described further herein. Accordingly, a “knowledge base” according to the present technology consists of criteria for the evaluation and selection of detection protocols.

In particular, the knowledge base comprises a body of IF . . . THEN rules that guide the selection of targets, primer pairs, assay conditions, and detection criteria for an assay to detect a nucleic acid. The expert system proposes solutions to the user, collects necessary data from the user, refines solutions when needed, and arrives at an appropriate assay for the user's desired test. The Examples provide an exemplary illustration of a user dialog with an embodiment of the technology comprising an expert system.

Although the disclosure herein refers to certain illustrated embodiments, it is to be understood that these embodiments are presented by way of example and not by way of limitation.

### EXAMPLES

### Example 1

During the development of embodiments of the technology, the following exemplary generic algorithm was developed to illustrate aspects of a user's interaction with the technology comprising an expert system. In particular, the exemplary generic algorithm provides an illustrative example of an application of the technology to answer the basis question: What nucleic acid(s) is/are in a given sample?

Generic Algorithm

| 1. | INPUT | |
|---|---|---|
| | USER INPUT: | |
| | (a) | What is the sample type (e.g., blood, sputum, urine, stool, |
| | | cerebral spinal fluid, culture, environmental, etc.)? |
| | (b) | What type of nucleic acid is to be detected (e.g., unknown, |
| | | human (forensic, biomarker), nonhuman (pathogen (bacterial, |
| | | viral, fungal, protozoal), virulence factors, drug resistance |
| | | markers, genotyping))? |
| 2. | SAMPLE PREPARATION | |
| | SYSTEM ACTION: | |
| | (a) | Select sample preparation protocol from the Sample Preparation |
| | | Protocol Database based on user input in 1(a) and 1(b). |
| | (b) | Perform the selected sample preparation protocol on the sample, |
| | | including pre-amplification (e.g., whole genome amplification, |
| | | TWGA, TGA) if it is a step of the selected sample preparation |
| | | protocol |
| 3. | AMPLIFICATION | |
| | SYSTEM ACTION: | |
| | (a) | Select reaction vessel combination from the Random Access |
| | | Primer Pair Library and/or synthesize primer pairs and |
| | | formulate corresponding reaction mixtures in one or more |
| | | reaction vessels based on user input in 1(b). |
| | (b) | Select amplification protocol from the Amplification Protocol |
| | | Database based on the reaction vessel combination selected |
| | | in 3(a). |
| | (c) | Perform the selected amplification protocol on at least a portion |
| | | of the nucleic acid sample from 2(b) to yield amplicons. |
| 4. | DATA GENERATION | |
| | SYSTEM ACTION: | |
| | (a) | Determine complete/partial base compositions and/or sequences |
| | | of the amplicons from 3(c), depending on the detection |
| | | system(s) included in the Amplicon Detection Module (including |
| | | any post-amplification amplicon processing steps (e.g., de- |
| | | salting)) to yield data. |
| 5. | DATA ANALYSIS | |
| | SYSTEM ACTION: | |
| | (a) | Interrogate the Base Composition Database (e.g., having |
| | | complete and/or partial base compositions) and/or the |
| | | Amplicon Sequence Database using the data from 4(a) to |
| | | yield output. Perform additional steps 2-5, if needed, |
| | | based on the output of a given step 5(a) in view of 1(b)). |
| | (b) | Provide the output to the user via the User Interface |

### Example 2

During the development of the technology provided herein, sample interactions of a user and the technology comprising an expert system were developed to illustrate certain aspects of the technology. The sample dialog is exemplary of a particular application of the technology to detect a pathogen from a wound culture. The actions performed by the system are exemplary of the expert system artificial intelligence component interacting with other system components to solve the User's problem.

| 1. | USER INPUT: | |
|---|---|---|
| | (a) | What is the sample type? Wound Culture |
| | (b) | What type of nucleic acid is to be detected? Pathogen |
| 2. | SYSTEM ACTIONS: | |
| | (a) | Select sample prep protocol from the Sample Prep Protocol |
| | | Database for Wound Culture and Pathogen user input in l(a) and |
| | | l(b). |
| | (b) | Perform the selected sample prep protocol on the sample, |
| | | including pre-amplification (e.g., WGA, TWGA, TGA) if it |
| | | is a step of the selected sample preparation protocol. |
| | (c) | Select reaction vessel combination from the Random Access |
| | | Primer Pair Library and/or synthesize primer pairs and |
| | | formulate corresponding reaction mixtures in one or more |
| | | reaction vessels based on generic Pathogen user input in |
| | | 1(a) and 1(b). |
| | For example, based on the User input the System selects reaction | |
| | vessels from the Random Access Primer Pair Library and/or | |
| | synthesizes primer pairs and formulates corresponding reaction | |
| | mixtures in one or more reaction vessels that include broad range | |
| | primer pairs for bacteria, virus, fungi, and protozoans. | |
| | (d) | Select amplification protocol from the Amplification Protocol |
| | | Database based on the reaction vessel combination selected in |
| | | 2(c). |
| | (e) | Perform the selected amplification protocol on a portion of the |
| | | nucleic acid sample from 1(b) to yield amplicons. |
| | (f) | Determine complete and/or partial base compositions and/or |
| | | sequences of the amplicons from 2(e), depending on the |
| | | detection system(s) included in the Amplicon Detection |
| | | Module (including any post-amplification amplicon |
| | | processing steps (e.g., de-salting)) to yield data. |
| | (g) | Interrogate the Base Composition/Amplicon Sequence Database |
| | | using the data from 2(f) to yield output. |
| | (h) | Provide the output from 2(g) to the user via the User Interface. |
| | For example, the data from 2(g) indicates that the nucleic acid | |
| | sample of 2(b) includes only bacterial nucleic acid. | |
| | (i) | Repeat 2(c)-(h) where the reaction vessel combination from the |
| | | Random Access Primer Pair Library and/or primer pairs and |
| | | corresponding reaction mixtures in one or more reaction vessels |
| | | are selected and/or synthesized and/or formulated to identify |
| | | the species of bacterial nucleic acid output in 2(g) to yield |
| | | bacterial species data. |
| | | For example, the bacterial species data from 2(i) indicates |
| | | that the nucleic acid sample of 2(b) includes only Klebsiella |
| | | pneumonia nucleic acid. |
| | (j) | Provide the output from 2(i) to the user via the User Interface. |
| | (k) | Repeat 2(c)-(h) where the reaction vessel combination from the |
| | | Random Access Primer Pair Library and/or primer pairs and |
| | | corresponding reaction mixtures in one or more reaction vessels |
| | | are selected and/or synthesized and/or formulated to identify |
| | | one or more characteristics of the species of bacterial nucleic |
| | | acid output in 2(i) to yield characterizing bacterial species |
| | | data. |
| | | For example, the Klebsiella pneumonia nucleic acid from 2(i) |
| | | indicates that the nucleic acid sample of 2(b) includes beta- |
| | | lactamase-mediated carbapenem resistance. |
| | (l) | Provide the output from 2(k) to the user via the User Interface. |

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in pharmacology, biochemistry, medical science, or related fields are intended to be within the scope of the following claims.

We claim:

1. A system for identifying a nucleic acid, the system comprising:
a) a random access primer component configured to provide a primer;
b) a nucleic acid amplification component configured to amplify a nucleic acid using the primer to generate an amplicon; and
c) an amplicon detection component configured to detect a property of the amplicon.

2. The system of claim 1 further comprising an expert system.

3. The system of claim 1 further comprising a conveyance component configured to convey the primer from the random access primer component to the amplification component and/or configured to convey the amplicon from the nucleic acid amplification component to the amplicon detection component.

4. The system of claim 3 further comprising a controller operably connected to one or more of the random access primer component, the nucleic acid amplification component, the amplicon detection component, and/or the conveyance component and configured to effect one or more of conveying the primers from the random access primer component to the nucleic acid amplification component, conveying the amplicon from the nucleic acid amplification component to the amplicon detection component, amplifying the nucleic acid with the nucleic acid amplification component, and/or detecting a property of the amplicon with the amplicon detection component.

5. The system of claim 1 wherein the random access primer component comprises a random access primer library and/or an oligonucleotide synthesis component.

6. The system of claim 1 wherein the nucleic acid amplification component comprises a thermocycler component.

7. The system of claim 1 wherein the amplicon detection component comprises a mass spectrometer component, a fluorescence detection component, and/or a nucleic acid sequencing component.

8. The system of claim 1 wherein the property of the amplicon is selected from the group consisting of presence/absence, mass, partial base composition, complete base composition, partial sequence, complete sequence, hybridization to a probe, electrophoretic mobility, length, hydrodynamic character, and restriction pattern.

9. The system of claim 1 wherein the random access primer component is configured to provide a primer pair.

10. The system of claim 1 wherein the random access primer component comprises 10 to 1000 primers.

11. The system of claim 1 wherein the nucleic acid amplification component is configured to pre-amplify a nucleic acid.

12. The system of claim 1 further comprising a sample preparation component configured to receive a sample and prepare the nucleic acid from the sample.

13. The system of claim 1 further comprising a database, wherein the database comprises a sample preparation protocol, a pre-amplification protocol, a primer datum, an amplification program, an amplicon detection protocol, and/or a reference amplicon property.

14. The system of claim 13 wherein the primer datum is a primer nucleotide sequence, a primer name, a primer location in the random access primer component, a primer melting temperature, and/or a primer target.

15. The system of claim 13 wherein the reference amplicon property is presence, mass, partial base composition, complete base composition, partial sequence, complete sequence, hybridization to a probe, electrophoretic mobility, length, hydrodynamic character, and/or a restriction pattern.

16. The system of claim 1 comprising a modular random access vessel.

17. The system of claim 1 comprising a modular random access vessel assembled into a reaction pathway.

18. The system of claim 1 comprising a vessel scaffold and/or a reagent storage component.

19. The system of claim 1 wherein the expert system comprises a knowledge base, wherein the knowledge base comprises rules for selecting an assay to detect a nucleic acid.

20. A method for the detection of a nucleic acid comprising: amplifying and detecting a nucleic acid molecule using the system of claim 1.
